(19) United States
(12) Patent Application Publication
(10) Pub. No.: US 2010/0266181 A1
(43) Pub. Date: Oct. 21, 2010

(54) DEVICE AND METHOD FOR PRODUCING A CT RECONSTRUCTION OF AN OBJECT COMPRISING A HIGH-RESOLUTION OBJECT REGION OF INTEREST

(75) Inventors: Steven Oeckl, Erlangen (DE); Theobald Fuchs, Nuernberg (DE); Tobias Schoen, Nuernberg (DE)

Correspondence Address: SCHOPPE, ZIMMERMANN , STOCKELER & ZINKLER;C/O KEATING & BENNETT, LLP, 1800 Alexander Bell Drive, SUITE 200, Reston, VA, 20191, US

(73) Assignee: Fraunhofer-Gesellschaft zur Foerderung der angewandten Forschung e.V.

(21) Appl. No.: 12/741,071

(22) PCT Filed: Apr. 30, 2009
(86) PCT No.: PCT/EP09/03149

§ 371 Date: Jun. 3, 2010

(30) Foreign Application Priority Data
Apr. 30, 2008 (DE) 102008021639.9

(51) Int. Cl. G06K 9/00; A61B 6/03

(52) U.S. Cl. 382/131; 378/5

(57) ABSTRACT

A CT reconstruction of an object including a high-resolution object region of interest may be produced in an artefact-free manner by producing a first projection data set of a first region of the object that encloses the object region of interest and includes at least one projection recording of a first resolution, and a second projection data set of the object region of interest including at least a second projection recording of a second, higher resolution. The first and second projection data sets may be combined, in accordance with a combination rule, so as to obtain a CT reconstruction of the first region of the object having the first resolution and of the object region of interest having the second, higher resolution.

Embodiments of the present invention relate to possibilities of creating a CT reconstruction of an object that has a high resolution in an object region that is of particular interest, said resolution being lower in other object regions which adjoin or comprise said object region of interest.

### BACKGROUND OF THE INVENTION

In the non-destructive testing of objects, or in the non-destructive examination of patients by means of X-ray computed tomography, the achievable resolution is limited essentially by two factors. They include, firstly, the finite expansion of the radiation source, i.e. of that area from which the radiation used for tomography is emitted (for example the focal spot of an X-ray tube), and, secondly, the finite expansion of the detector elements. With a finite expansion of one of these two components, the idealized way of looking at an idealized “X-ray beam” of an infinitesimal expansion, which perspective underlies many reconstruction algorithms, is no longer fulfilled by the object to be examined.

The pixels used for sampling the individual projection images in an X-ray-sensitive detector, such as, for example, those of an electronic flat-panel image converter (for example of a CCD comprising a radiation-converting coating, or of a directly converting semiconductor detector, or the like) naturally have an intermediate distance that is finite in each case, whereby the resolution is also limited.

Since this typically corresponds to the practical circumstances, it shall be assumed below, without prejudice to the generality, that the expansion of the radiation source is smaller than the limitation of the resolution that is caused by the finite distance of the detector pixels.

In order to be able to reconstruct in an artefact-free manner, by means of standard algorithms, a series of X-ray projection images, i.e. a plurality of recordings, or X-rays, obtained from different perspectives by means of an extensive one- or two-dimensional detector, the object may be fully contained within the horizontal extensions in each of the recorded projections (if the object projects beyond the detector at the top and/or at the bottom, this will not lead to any artefacts in the reconstruction). In other words, for three-dimensional reconstruction (CT reconstruction), the object may be fully imaged in the horizontal extension on each two-dimensional shadow image (projection). Therefore, the horizontal extension describes, in this context, that orientation of the object relative to which the perspective is changed by the rotation. In the vertical direction which is perpendicular thereto, the object may be imaged in an incomplete manner. With a point-shaped radiation source and detector having finite dimensions, the object consequently cannot be positioned at any distance from the detector, since otherwise the geometrical projection of the object would protrude beyond the detector. The more severe the violation of the condition of fully imaging the object in each projection, the more intense the interferences in the CT sectional images or in the three-dimensional reconstructions of the object examined will be, said interferences being caused by the image reconstruction algorithm. The share of artefacts in the reconstructed layers or models thus increasingly impairs the diagnostic conclusion that can be drawn from the images, until, in extreme cases, said images contain no more useful information.

On the one hand, the resolution is thus limited by the apparatus used and, in particular, by the resolution of the sensor used. On the other hand, the condition of completely imaging the object in the individual projections, which condition is set up by the image reconstruction algorithm, limits the spatial resolution since, in this manner, the optical magnification of the object to be examined—caused by geometric variations of the distances between the detector and the object—on the detector is capped.

This is relevant particularly to geometrically expanded objects, such as in the non-destructive testing of the material of motors or similarly large components, wherein fine details can no longer be resolved with the available sensor resolution when, as was described above, the entire object is to be imaged, in each projection view, onto the sensor having a finite size.

The possibility of obtaining a complete set of projection data in a magnified view by not imaging the complete object per projection is limited since, in this case, the image reconstruction algorithm produces considerable artefacts. This is due to the fact that the image reconstruction algorithm depends on containing complete information about the object from all perspectives. However, this is not the case if the edge of object is not imaged in each the projections. However, this edge contributes, with a view rotated by 90°, to the absorption coefficient and, thus, to the entire X-ray absorption, so that the CT reconstruction algorithm produces considerable artefacts in reconstruction, whose sizes increase as the proportion of the region that is not imaged at the edge of the object increases.

Therefore, it is useful to be able to examine partial regions of an object by means of CT methods in a manner that is complete and free from artefacts, even with objects that have large geometrical expansions.

Some embodiments of the present invention enable highly detailed CT imaging of an object region of interest within an object in that, initially, a first projection data set of the object is produced at a first, low resolution, and in that subsequently, a second projection data set, which comprises only the object region of interest, is detected at a second, higher resolution. By combining the first and second projection data sets thus obtained in accordance with a combination rule, a CT reconstruction of the first region of the object comprising the first resolution may be obtained, it being possible to reconstruct that object region of interest that is arranged within or at the edge of the first region at the second, higher resolution. In this context it is possible, in particular, to reconstruct, by detecting the object twice, a complete data set that remains free from image artefacts within the object region of interest.

In accordance with some embodiments, the first and second regions are recorded simultaneously in that, for example, two detectors of different geometric expansions and spatial resolutions are used. The high-resolution detector may be arranged upstream from the low-resolution detector, for example, it being possible to substitute those regions of the low-resolution detector that are shadowed by the high-resolution detector by using the image data of the high-resolution detector to supplement the missing data in the image of the low-resolution detector.

In some embodiments, the object is moved, or the distance between the detector and the object is varied, so that the object may be recorded by means of a detector with different magnification factors, so as to obtain, by subsequently combining the two projection data sets, the representation of the object region of interest (ROI=region of interest), said representation having a high resolution and being free from artefacts.

In addition, in accordance with some embodiments, the concept of duplicate recording or of duplicate production of projection data sets of different resolutions may also be implemented in connection with different X-ray sources or with X-radiation of different wavelengths. Since the absorption cross-section of X-radiation is dependent on the material and energy, a maximum image contrast may be achieved, depending on the object investigated, for different energies of the X-radiation used. Thus, if the object to be examined contains different materials, different X-ray energies may result in that the achievable resolution is identically high for both materials despite the different absorption properties.

In the context of the present application or invention, the term resolution is therefore not only to be understood as spatial resolution, but as a term describing how much information may be obtained from the data or the individual projection recordings. An illustrative example of this may be the fact that, even though a detector with randomly high spatial resolution is used, the information obtained is only approximately zero if X-radiation of such low energy is used that it is almost entirely absorbed by the object. In the extreme case of very high X-ray energy, where hardly any absorption takes place, this is also true, of course.

If two projection recordings with different resolutions are produced, different possibilities or combination rules will result in accordance with which both projection recordings, or the different projection data sets consisting of several projection recordings, may be combined such that an image which is free from artefacts and has the high resolution of the second projection recording will result in the object region of interest.

A second possibility of combination and an associated second combination rule result when the data in the second projection recording are processed such that only information about the high-frequency image portions within the object region of interest is taken from said data. The low-frequency image information (low spatial frequencies) is already contained in the first projection recordings. If this pre-processing of the second projection data set is performed such that all of the image information or almost all of the image information above a cutoff frequency, which is defined by the maximally imageable position information in the lower-resolution images of the first projection data set, is contained therein, both projection data sets may be combined, by simply adding the corresponding recordings, such that the resolution of the second projection recordings is available within the object region of interest without artefacts or similar image interferences resulting from the separate pre-processing.

In some embodiments, pre-transformation is used for the second projection recordings so as to extract those higher-frequency image portions associated with the object region of interest which correspond, in a retro-projected image, to a wavelet decomposition of the recording. Due to the positional- and frequency-locality property of the wavelet base functions, the above criterion can be ensured. This means that the representation or wavelet decomposition can be selected such that the wavelets contribute to the reconstruction only within the object region of interest, but describe, within said object region of interest, the relevant information, namely the high-frequency image portions. In this manner, it is possible to obtain combination recordings which are provided, within the object region of interest, by the desired high resolution in an artefact-free manner. In particular the high-frequency and low-frequency image portions may be processed and reconstructed separately, it being possible to combine the representations after the reconstruction by means of addition.

It is readily possible for a person skilled in the art to draw, from the wavelet representation of the reconstructed recordings that is to be achieved, the conclusion as to which filters and/or operations of a pre-transformation are to be applied to the individual projection recordings.

### SUMMARY

According to an embodiment, a method of producing a CT reconstruction of an object including a high-resolution object region of interest may have the steps of: producing a first projection data set of a first region of the object that encloses the object region of interest and includes at least one projection recording of a first resolution; producing a second projection data set for the object region of interest that includes at least one second projection recording of a second, higher resolution; pre-transforming the second projection data set to acquire a pre-transformed second projection data set including, within the object region of interest, only image frequencies above a predetermined cutoff frequency, which is equal to or smaller than an upper imaging frequency, defined by the first resolution, within the first projection data set; and combining the first and the pre-transformed second projection data sets in accordance with a combination rule so as to acquire a CT reconstruction of the first region of the object including the first resolution, and of the object region of interest including the second, higher resolution.

According to another embodiment, a device for producing a CT reconstruction of an object including a high-resolution object region of interest may have: a detector configured to produce a first projection data set of a first region of the object that encloses the object region of interest and includes at least one projection recording of a first resolution; produce a second projection data set of an object region of interest including at least a second projection recording of a second, higher resolution; and a combiner configured to pre-transform the second projection data set to acquire a pre-transformed second projection data set including, within the object region of interest, only image frequencies above a predetermined cutoff frequency, which is equal to or smaller than an upper imaging frequency, defined by the first resolution, within the first projection data set, and to combine the first and the pre-transformed second projection data sets in accordance with a combination rule so as to acquire a CT reconstruction of the first region of the object including the first resolution, and of the object region of interest including the second, higher resolution.

According to another embodiment, a computer program has a program code for performing the method of producing a CT reconstruction of an object including a high-resolution object region of interest, wherein the method may have the steps of: producing a first projection data set of a first region of the object that encloses the object region of interest and includes at least one projection recording of a first resolution; producing a second projection data set for the object region of interest that includes at least one second projection recording of a second, higher resolution; pre-transforming the second projection data set to acquire a pre-transformed second projection data set including, within the object region of interest, only image frequencies above a predetermined cutoff frequency, which is equal to or smaller than an upper imaging frequency, defined by the first resolution, within the first projection data set; and combining the first and the pre-transformed second projection data sets in accordance with a combination rule so as to acquire a CT reconstruction of the first region of the object including the first resolution, and of the object region of interest including the second, higher resolution, when the program runs on a computer.

Other elements, features, steps, characteristics and advantages of the present invention will become more apparent from the following detailed description of the preferred embodiments with reference to the attached drawings.

### BRIEF DESCRIPTION OF THE DRAWINGS

Embodiments of the present invention will be detailed subsequently referring to the appended drawings, in which:

FIG. 1 shows a setup of a CT system;

FIGS. 2A and 2B schematically shows the production of two projection data sets of different resolutions;

FIG. 3 schematically shows an alternative possibility of simultaneously producing two projection data sets of different resolutions;

FIG. 4 shows an embodiment of a combination rule for combining the projection recordings of different resolutions so as to obtain a CT reconstruction of the object region of interest;

FIG. 5 depicts an alternative embodiment of a combination rule so as to obtain a CT reconstruction from two projection data sets of different resolutions;

FIG. 6 shows a further embodiment of a combination rule so as to obtain a CT reconstruction from two projection data sets of different resolutions; and

FIG. 7 shows a further embodiment of a combination rule.

### DETAILED DESCRIPTION OF THE INVENTION

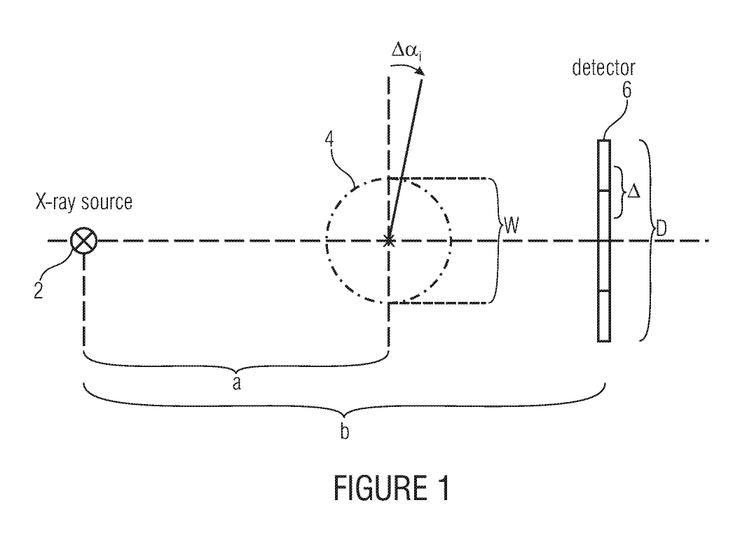

FIG. 1 schematically shows the fundamental elements and their relative arrangement of a CT measurement setup, which need to be understood as the basis for the following discussion of several embodiments of the invention. FIG. 1 shows an X-ray source 2, assumed (in an idealized manner) to be point-shaped, by means of which an object arranged within a measuring field 4 is imaged to a detector 6. Those factors which, with a point-shaped X-ray source 2, limit the spatial resolution achievable are the distance b of the X-ray source 2 from the detector 6, the distance a of the X-ray source 2 from the center (position of the axis of rotation) of the measuring field 4, the size of the object W (here assumed to be a diameter of the measuring field), and the distance Δ of two adjacent pixels or detector elements of the detector 6, whose entire geometric expansion within the dimension contemplated is to be D.

As was already described above, for the application of standard CT image reconstruction methods, the object (in this case replaced by the spatial expansion W of the measuring field 4) should be fully contained, in the horizontal expansion, within the individual projection recordings of the detector 6. A projection data set consisting of several projection recordings is typically obtained in that either the object and/or the measuring field 4 is rotated relative to the arrangement of the X-ray source 2 and the detector 6, it being possible, in principle, to freely select the angle increments Δα₁ of the rotation, as will be explained below. Alternatively, the X-ray source 2 and the detector 6 may be rotated about the center of rotation (center of the measuring field 4), as, for example, in a computer tomograph for application in human medicine. If the above requirement of complete imaging of the object in the projections is met, this will mean that an effective spatial resolution Δ_{effective} will depend on the size of the object and, thus, on the maximally applicable magnification, since effective sampling (within the object) by a detector (pixel distance Δ) decreases as the magnification factor increases:

Δ_{effective}=Δ/M (1)

The magnification factor M in this context is defined by the distances of the axis of rotation (which typically has the object arranged centrally thereon) from the detector and/or from the source

$$M = \frac{b}{a} , \qquad ( 2 )$$

wherein b is the distance of the source from the detector, and a is the distance of the source from the center of rotation.

Let W be the diameter of the measuring field and, thus, the maximum object size that guarantees that each projection is fully imaged to the detector. The upper limit for the magnification results from this as

$${M}_{max} = \frac{D}{W} , \qquad ( 3 )$$

wherein D is the width of the input face of the detector, which is typically specified by the design of the flat-panel image converter installed within the CT system and which cannot be adapted to different problems.

For a flat-panel image converter with N_{D} detector elements per column or row (the validity of this consideration not being limited to square detectors), the following applies:

$$\begin{matrix}D = {N}_{D} \cdot Δ \\ and , thus & ( 4 ) \\ {Δ}_{effective , min} = Δ / {M}_{max} = \frac{W}{{N}_{D}} & ( 5 )\end{matrix}$$

This means that in the event of complete imaging of the object in the above-described sense, the resolution will be effectively limited by W/N_{D} even with a sufficiently small focal spot. For example, a cylindrical casting having a diameter of 200 mm can be imaged, while using a 1,024-column detector, no better than at a resolution of approx. 195 μm in the sectional planes.

On the basis of the preceding considerations, it is impossible to select a magnification factor that is sufficiently large that, for each projection, only an inner sub-region of an object is recorded within the measuring field 4, but is recorded with a larger magnification to make up for it, since this would lead to intense image artefacts which in extreme cases would render useful interpretation of the recordings impossible.

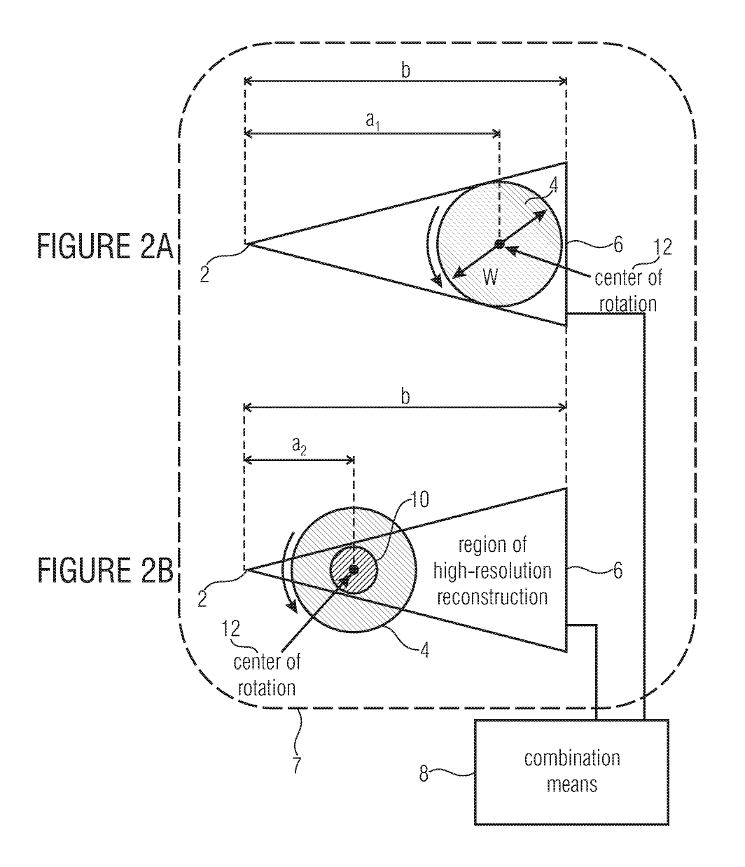

FIG. 2 shows an embodiment of the invention wherein a first projection data set with a first region of the object, which comprises an object region of interest, is produced at a first resolution, whereas a second projection data set for the object region of interest is produced at a second, higher resolution. This enables combining the two projection data sets by means of a suitable combination rule so as to arrive at a CT reconstruction of the object, or of that part of the object that is of interest, said CT reconstruction having a high resolution in the object region of interest without having any artefacts.

FIG. 2 draws on the same symbols and terminology as have already been introduced in FIG. 1, so that repeated explanation of the corresponding components can be dispensed with.

Generally, within the context of the present description, elements that are identical or similar in function are provided with the same reference numerals, it being possible to interchange their descriptions with regard to the individual figures.

FIG. 2a shows the situation of how a projection data set of the entire object (measuring field) 4 is initially created by means of one and the same detector 6, said projection data set having a first resolution determined by the above-described geometric conditions.

FIG. 2b illustrates how a second projection data set may be obtained, using the same detector 6, for an object region of interest, which here is arranged at the center of the measuring field 4 without prejudice to the generality. Alternative embodiments or alternative applications of the concept may naturally also image, at a high resolution, such object regions that are not located at the center of rotation.

As is depicted in FIG. 2b, the distance between the object 4 (or between the center of rotation 12) and the detector 6 is varied in order to change the magnification factor. To this end, for example, the detector may be moved away from, or toward, the object so as to change the magnification. Said magnification corresponds, in a figurative sense, to a detector or to two separate detectors, as will be described below, and which comprise a high-resolution region within a low-resolution region.

The detector is connected to a combination means 8 that enables combining the projection data sets, as will be set forth later on.

In other words, FIGS. 2a and 2b show a repeated measurement of the same object with different magnification factors so as to enable high-resolution reconstruction without any artefacts within an object region 10 of interest. For the first measurement, the magnification is selected such that the object may be fully imaged in all projections and may be reconstructed in an artefact-free manner with a second resolution. The second measurement is performed with a magnification factor that may be selected with respect to the desired resolution to be achieved without it being necessary to consider complete detection of the entire object.

FIGS. 2a and 2b therefore show two possible operating modes of a detection means 7, which enables creating a first projection data set having a first resolution, and a second projection data set having a second, higher resolution.

The combination means 8 enables combining the first and second projection data sets in accordance with a combination rule so as to obtain a CT reconstruction of the first region 4 of the object with the first resolution, and of the object region 10 of interest with the second, higher resolution.

Both data sets may be combined by suitable means such that within the object region of interest (ROI), in this case, for example, the central region, the desired resolution is achieved without the image quality in the volume data set, which represents the entire object, comprising, after the CT reconstruction, artefacts that result from the incompleteness of the data.

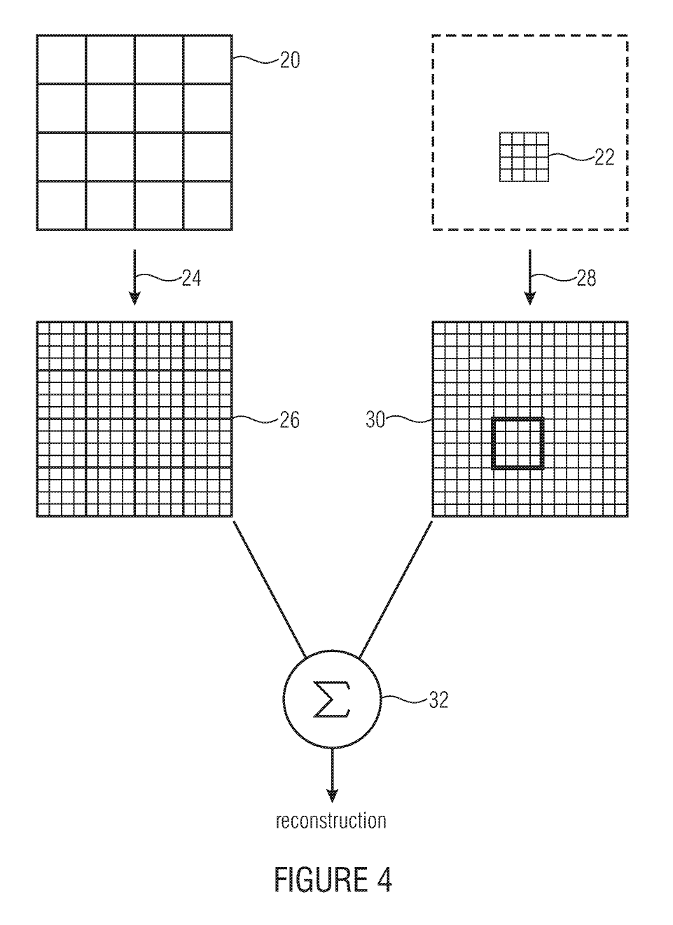

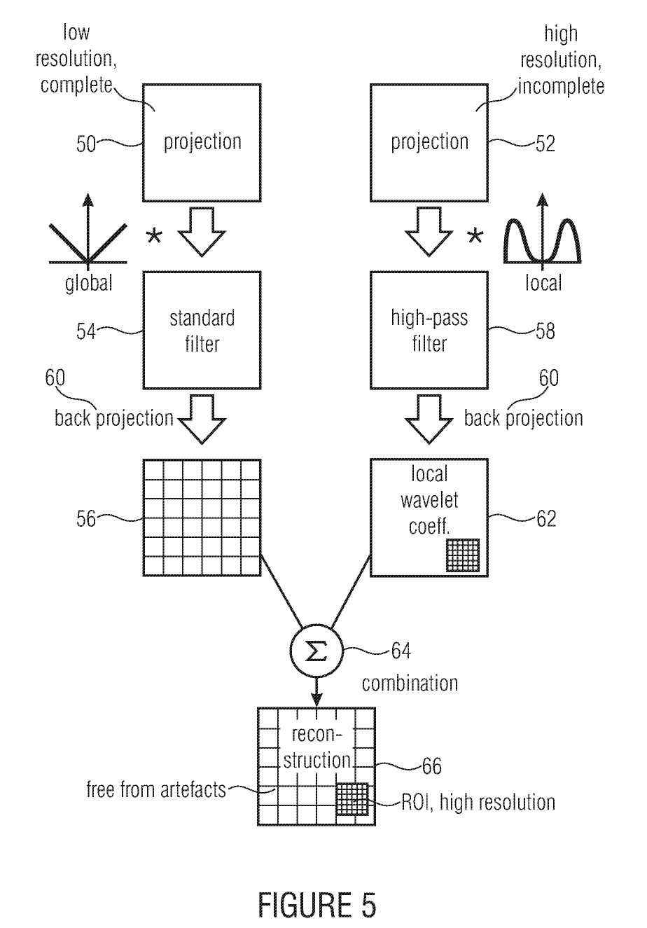

Embodiments of suitable combinations or suitable combination rules (means for combining) will be explained below in more detail with reference to FIGS. 4 and 5.

In the approach suggested in FIGS. 2a and 2b, there is also the possibility, with regard to the angle increments at which the rotation is performed, of optimizing the number of the projections that may be used with regard to the resolution by selecting the angle increments in a suitable manner, and, thus, to increase the efficiency and to save measuring time.

This means that with a flat-panel image converter of a specific size, said converter being predefined by the CT system available, the user-selectable parameter regarding the number of angular positions on the full circle from which projection images are acquired may be selected.

With an assumed angle increment of Δα, radial sampling at the distance R_{M} from the center of rotation 12 results directly from the arc length:

Δᵣ=R_{M}·Δα (6)

The angular increment follows directly from the number of angular positions on the full circle:

$$Δ α = \frac{2 π}{{N}_{α}} \qquad ( 7 )$$

If radial sampling at the edge of the measuring field (the maximum distance of two rays in successive projections) is equated with the sampling distance Δ_{effective} within the projection from (1), the following results for the number of projection angles:

$$\begin{matrix}\frac{D}{{N}_{D} \cdot M} = {P}_{M} \cdot \frac{2 π}{{N}_{α}} \\ or & ( 8 ) \\ {N}_{α} = {N}_{D} \cdot \frac{W \cdot π}{D} M . & ( 9 )\end{matrix}$$

In the simplest case, the number of the acquired projections will be identical in both measurements described above. However, in order to reduce the overall time that may be taken to perform both measurements and, thus, to increase the test efficiency of a computer tomography system designed in accordance with the present invention, the number of projections may be reduced, in the first measurement (which fully images the object), to such an extent that the minimum requirements for angle sampling are met only within the sub-region to be reconstructed at a high resolution.

This means that the fact that there is an interest only in the image information within the object region of interest can additionally be accounted for in that, even in the lower-resolution recording, the information having the resolution that is maximally possible in view of the reduced resolution is recorded only for the object region of interest.

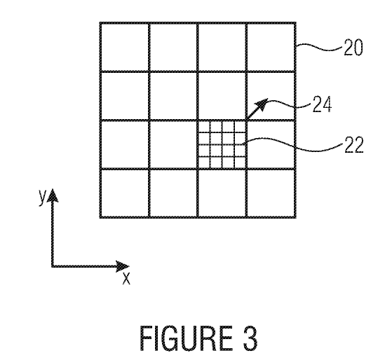

FIG. 3 shows an alternative embodiment for simultaneously producing a first projection data set and a second projection data set by means of two different detectors having different resolutions. For clarity's sake, only the schematic, two-dimensional top views of a first detector 20 and of a second detector 22 are depicted here.

The first detector 20, which has a lower spatial resolution, may be arranged, for example, upstream or downstream from the second detector 22 having the higher spatial resolution. If useful for the image reconstruction or for combining the images, the image information which is missing for the second detector 22 by shadowing on the projection recordings of the first detector 20, may be readily substituted by the image information of the second detector 22. An example of such a substitution would be, for example, to sum the intensities of the high-resolution pixels in a weighted manner or to combine them in any other manner so as to achieve an intensity value corresponding to a detector pixel of the first detector 20.

The arrangement shown here by way of example is not limited to square geometries; rather, any detector shapes may be randomly combined with one another. Also, the second detector 22 may be arranged such that it does not fully cover an even number of pixels of the first detector 20, as is indicated here for simplicity's sake.

In addition, in accordance with some embodiments, the second detector 22 may be arranged to be movable relative to the first detector 20, as is indicated here, for example, by a directional arrow 24, so that the second detector 22 may be moved in two dimensions (x and y directions) relative to the first detector 20. With setups that are not fully rotationally symmetric, i.e. wherein the second detector 22—as is already shown here—is not arranged at the center of the first detector 20, this may be used for having the second detector follow the region of interest or the object region of interest upon rotation of the object. In addition, the second detector 22 may also be movable in three dimensions so as to be able to vary, additionally, the effective resolution of the second detector 22 independently of the first detector 20.

Additionally, a second detector arranged upstream from a first detector acts as a prefilter for X-radiation, and ensures, at a constant tube voltage or X-ray energy, that the beam qualities and, thus, the contrast with which different materials are imaged differ between the two projections of different spatial resolutions. In this manner, a two-spectra data set may additionally be recorded which can be evaluated using the known methods of material analysis.

FIG. 4 shows a first example of a combination rule that may be used for obtaining CT reconstructions of an object wherein an object region of interest is imaged at a high resolution without producing any artefacts. The representation in FIG. 4 schematically depicts the detectors 20 and 22 as were introduced in FIG. 3. By means of the first detector 20, a first projection recording having a first, low resolution is produced, and with the second detector 22, a second projection recording having a second, higher resolution is produced. The individual projection recordings of the object taken from the same perspective may be combined, as is indicated in FIG. 4, before being handed over to a standard CT image algorithm. To this end, as is depicted in FIG. 4, the first projection recording of the first detector 20 is resampled at the resolution of the second detector 22. This may be performed in a resampling step 24. The image information comprising the higher resolution may be produced, for example, by means of interpolation techniques from the image information of the first projection recording of the first detector 20 so as to produce a first intermediate image 26.

Alternatively or additionally, as is indicated by the supplementation step 28, a second intermediate image 30 may be produced from the second projection recording in that image information outside the object region of interest recorded directly by means of the second detector 22 is supplemented with a resolution corresponding to the resolution of the second detector 22. The image information, useful for supplementation, for generating the second intermediate image may be obtained from the image information of the first projection recording 20. In both cases it is useful, in order to produce the intermediate image (third intermediate image), which may be handed over to a standard CT algorithm, to suitably combine the first projection recording 20 and the second projection recording 22. If the first intermediate image 26 is directly combined with the second projection recording 22, the above may be effected by a summation 32, for example. Alternatively, the image information that is already contained within the second projection recording 22 may be fully removed from the interpolated intermediate image representation 26 of the first projection recording prior to summation, so that there will be no “overexposure”, in terms of intensity, of the image region in question.

In other words, FIG. 4 shows a method for combining the two data sets in that the data that are missing in the high-resolution measurement are supplemented, in the regions of the lacking information, by the complete data from the complementary measurement. In this context, only corresponding re-scaling of the complete data to a finer sampling raster may be performed, the densities of the sampled projection values having the same mutual ratio as the magnification factors. The high-resolution projections are supplemented, at the edge of the real image, by virtual detector columns, whose entries are taken from the complete data from the corresponding perspective with regard to the beam through the center of rotation.

FIG. 5 illustrates an alternative possibility of combination, i.e. an alternative implementation of a combination rule so as to combine the first projection data set and the second projection data set, or the information contained within the data sets, so as to obtain a direct reconstruction of high-resolution image data within that sub-region of the object that is measured in a magnified manner.

In this manner, a back projection of the projection data set 50 having the first, low resolution may initially be performed separately from a back projection of the second projection data set 52 having the second, higher resolution. Provided that in the first projection data set 50, the object comprising the object region of interest is fully imaged, a CT reconstruction of the first projection data set may be performed using conventional CT image reconstruction algorithms. They include, for example, applying globally effective filters to the individual projection recordings so as to obtain filtered projection recordings 54. By means of back-projecting the filtered projection recordings, a low-resolution CT reconstruction 56 of the data may occur. Irrespective thereof, the projection recordings associated with the second, higher-resolution projection data set 52 may be filtered using a location- and frequency-local filter (corresponding to wavelet decomposition, for example) on the basis of said second projection data set 52, so as to obtain a high-frequency-filtered representation of the projection recordings of the higher resolution 58.

By means of the back projection 60, an intermediate CT reconstruction of the object is then produced within the object region of interest, which intermediate CT reconstruction contains only information about high-frequency image portions (intermediate back projection or intermediate reconstruction 62). If the intermediate CT reconstruction contains only image portions of a frequency that is above a cutoff frequency defined by the finite resolution of the first projection recordings, the intermediate CT reconstructions 56 and 62, which have been produced separately, may be combined by means of a combiner 64 so as to obtain, after the combination step has been effected, a CT reconstruction 66 of the object wherein the object region of interest comprising the second, higher resolution is contained without image artefacts having been produced by the image reconstruction. During combining 56 and 62, 56 is possibly treated with an operator associated with the filters, applied to the projection, for reconstructing the low-frequency portions, and 62 is treated with an operator associated with the filters, applied to the projection, for reconstructing the high-frequency portions, before the two intermediate CT reconstructions which have been pre-processed in this manner are added.

In other words, FIG. 5 depicts an example of a direct method of reconstructing high-resolution image data within the sub-region, measured in a magnified manner, of the object by applying wavelet-based reconstruction. To this end, the property of the wavelet coefficients of being local both in the position space and in the frequency space and, thus, contribute only to a limited frequency and spatial section in the image is exploited. Therefore, in a wavelet reconstruction of the complete data set within the central region of the object, the high-frequency contributions from the high-resolution data set may be casually supplemented, so that in one go the object may be reconstructed free from artefacts induced by incompleteness of the projections, and can be reconstructed at the desired resolution within the central sub-region.

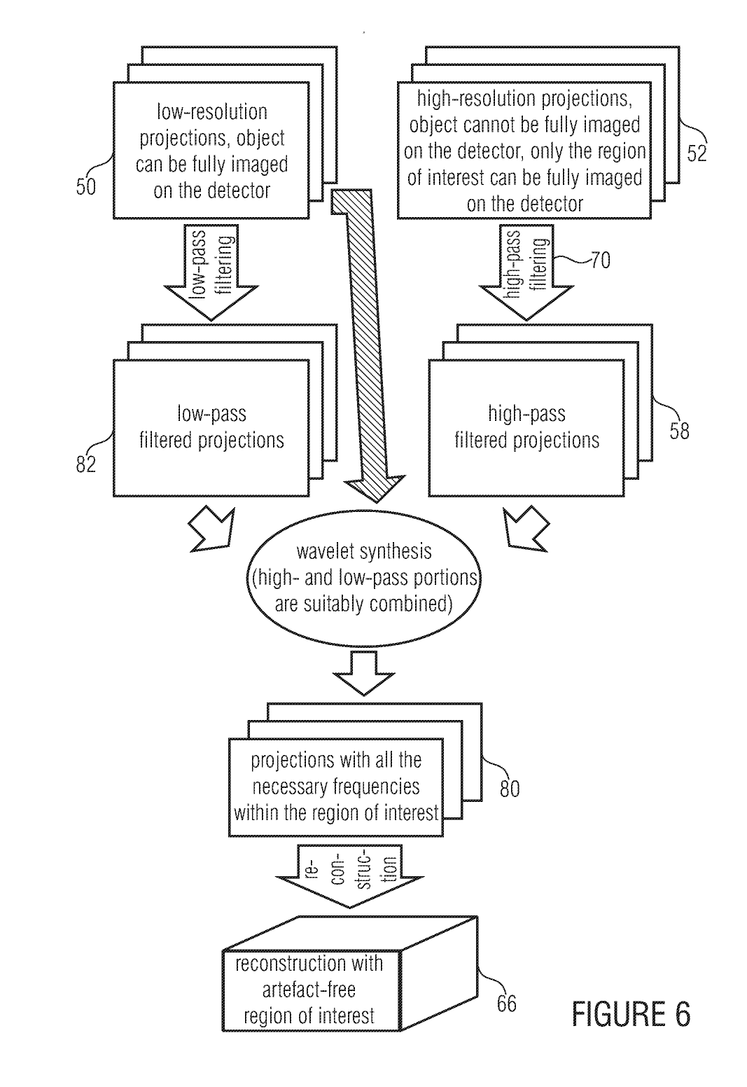

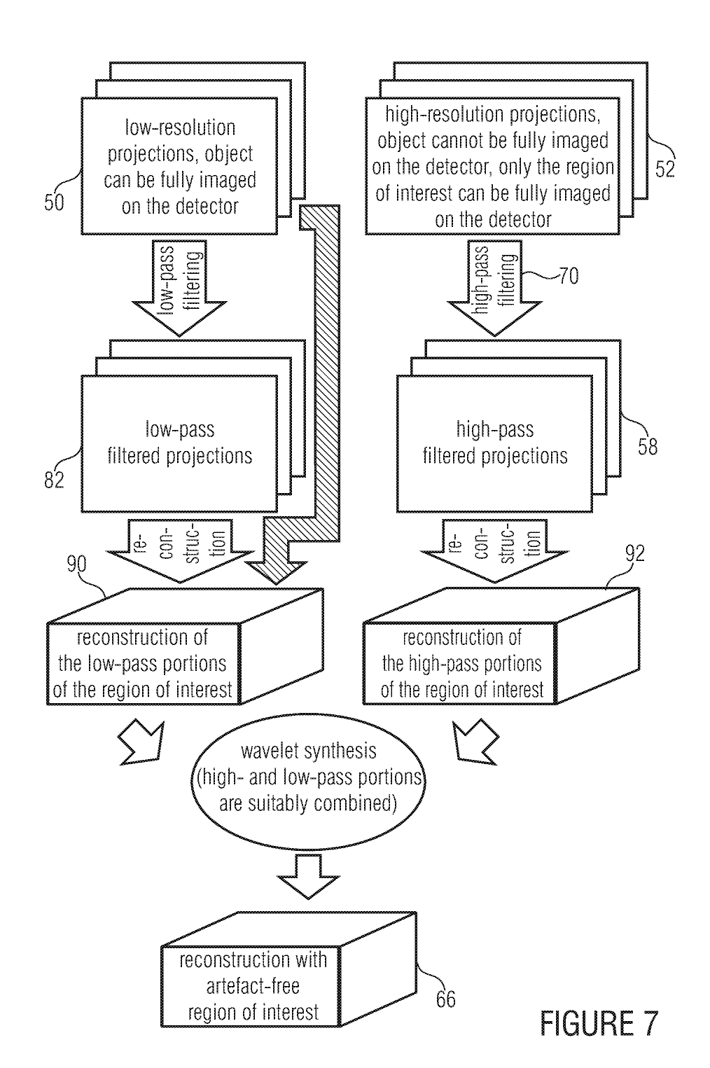

FIGS. 6 and 7 illustrate two alternative possibilities of how a 3D reconstruction, i.e. a CT reconstruction of an object including the object region of interest that is to have a high resolution, may be produced in accordance with the invention. Firstly, the projection data sets having different resolutions may be combined prior to the actual CT reconstruction (FIG. 6). Secondly, separate CT reconstruction may be performed on the basis of the first projection data set and of the pre-transformed second projection data set, before the two intermediate CT reconstructions thus obtained are combined so as to obtain the final CT reconstruction of the object.

With the CT reconstruction, any CT reconstruction algorithms may be used as have been known so far or will be developed in the future.

FIG. 6 shows an example of how, by combining the projection data sets prior to the actual CT reconstruction, a CT reconstruction of the object may be obtained which has a high spatial resolution within the object region of interest. A first projection data set 50 of the object is initially produced which comprises at least a first projection recording having a first resolution. In addition, a second projection data set 52 is created, prior to this, at the same time, or later on, for the object region of interest, which comprises at least a second projection recording of a second, higher resolution (higher than the resolution of the first projection data set). Thus, the projection recordings of the first projection data set having the lower resolution contain spatial frequencies or image frequencies up to a predetermined cutoff frequency limited by the resolution. The projection recordings of the second projection data set also contain, due to the higher spatial resolution, spatial frequencies or image frequencies above said cutoff frequency.

In a pre-transformation step 70, the second projection data set, or the projection recording of the second projection data set, is pre-transformed so as to obtain a pre-transformed second projection data set 58 which merely comprises image frequencies above a predetermined frequency. This frequency may be, e.g., the cutoff frequency of the first projection data set, or any other frequency matched or tuned to the cutoff frequency. The pre-transformation may be high-pass filtering, for example.

However, any other pre-transformations which result in that only image frequencies above a predetermined cutoff frequency are contained within the pre-transformed second projection data set are also applicable. Contained merely within the second projection data set evidently also means that the lower frequencies need not be fully removed, but that it is sufficient to suppress them with regard to the high-frequency image portions.

Parallel therewith, the first projection data set may optionally be subject to low-pass filtering adapted to the cutoff frequency of the high-pass filtering of the second projection data set. Alternatively, said low-pass filtering may also be dispensed with, however, since, due to the finite detector resolution with which the first projection data set was obtained, the data of the first projection data set per se have a cutoff frequency above which no image frequencies can be contained within the projection recordings.

In accordance with some embodiments of the present invention, while combining the first and the pre-transformed second projection data sets in accordance with a combination rule, a CT reconstruction 66 is eventually produced which in the first region of the object comprises the first resolution, and in the object region of interest comprises the second, higher resolution. In accordance with the embodiment described in FIG. 6, a third projection data set 80 is initially formed, for this purpose, by combining the first projection data set 50 and the pre-transformed second projection data set 58. As was already mentioned, it is also possible, alternatively, to produce the combination by means of a representation, which is also pre-transformed, of the first projection data set. What is essential is that the combination is formed such that the third projection data set within the object region of interest contains image frequencies that are both above and below the predetermined cutoff frequency on which the pre-transformation of the second projection data set is based. One possibility of performing this combination is a wavelet synthesis of the first projection data set and of the pre-transformed second projection data set so as to arrive at the third projection data set.

The CT reconstruction 66 is then obtained in that a CT reconstruction algorithm is applied to the third projection data set.

In other words, in FIG. 6, the projection data sets are not combined by means of interpolation/extrapolation but, in the projection data set having the high magnification or the high resolution, the high frequencies are extracted by means of suitable filtering, it being optionally possible, additionally, within the projection data set having the lower magnification, to extract the low frequencies by suitable filtering. These high- and low-frequency portions may then be merged into a new projection 80 by means of a wavelet synthesis or any other suitable measure, said new projection 80 containing all of the frequencies in the region of interest (the object region 10 of interest). The frequencies that can, or should, be extracted from the individual projection data sets are predefined by the boundary conditions and may be varied freely within wide limits. A factor to be taken into account may be the magnification factor, for example, which results from that, e.g., the region of interest having the maximum magnification is to be imaged with a given detector geometry. I.e., the magnification factor ensures that the corresponding frequencies are actually contained in the projections. Alternatively or additionally, more than two projection data sets, as is illustrated in FIG. 6, may be formed and be combined in an equivalent manner.

The method illustrated in FIG. 6 has the advantage that no artefacts occur that might be caused by numerical interpolation/extrapolation. This is the case with conventional methods wherein the projection data sets having the large magnification are continued merely on the basis of the projection data sets having the low magnification, for which purpose they need to be interpolated. Due to the useful interpolation/extrapolation, said methods result in numerical inaccuracy when the projections are merged. In addition, it cannot be ensured that all of the frequencies are fully contained within the merged projection. However, this is advantageous for artefact-free reconstruction. However, the method described in FIG. 6 and, subsequently, in FIG. 7 ensures that all of the frequencies that may be used are present within the region of interest, which enables artefact-free reconstruction within the region of interest.

FIG. 7 shows an alternative approach to producing a CT reconstruction 66, which in the object region of interest has a higher resolution than in those regions of the object which surround said object region. With regard to producing the projection data sets and their possible pre-transformations, the method described in FIG. 7 corresponds to the method described in FIG. 6, so that with regard to the description of these steps, reference shall be made to the corresponding description concerning FIG. 6.

The difference consists in that on the basis of the first projection data set and the pre-transformed second projection data set, separate intermediate CT reconstructions are initially produced which are subsequently copied to become the final CT reconstruction 66. In other words, a first intermediate CT reconstruction 90 is produced from the first projection data set 50 or its pre-transformed representation 82. By analogy therewith, a second intermediate CT reconstruction 92 is produced from the pre-transformed second projection data set 58, said second intermediate CT reconstruction 92 obviously comprising, even in the 3-dimensional representation, only image frequencies above the predetermined cutoff frequency. The CT reconstruction is then obtained by combining the first intermediate CT reconstruction 90 and the second intermediate CT reconstruction 92. In accordance with an embodiment of the invention, these two intermediate CT reconstructions 90 and 92 are combined by means of a wavelet synthesis.

I.e., the method suggested in FIG. 7 is also based on employing different projection data sets. The projection data set having the high magnification or resolution (the second projection data set 52) may be filtered such that the reconstruction 92 resulting therefrom only contains the high frequencies that may be used for a wavelet synthesis. Optionally, the projection data set having the lower magnification may be filtered such that the reconstruction resulting therefrom contains only the low frequencies that may be used for a wavelet synthesis if this property does not result from the finite detector resolution itself. The region of interest may be composed, in an artefact-free manner, of these two reconstruction data sets (for example by means of a wavelet synthesis).

The method described in FIG. 7 has the advantage, for example, that in the combination of intermediate reconstructions that have already been performed, fewer projection recordings may be used for the projection data set for reconstructing the low frequencies from the projections having a lower resolution. This is so precisely because initially, intermediate reconstructions are produced, i.e. the combination is not effected on the basis of the projection recordings already. If one uses dyadic discretization, for example, it is sufficient, with the lower magnification, to measure only half as many projections as with the higher magnification. Firstly, this saves measuring time and, secondly, it reduces the dose of radiation the object to be examined is exposed to.

FIGS. 4 to 7 describe only two examples of a suitable connection or combination of the first projection data set and of the second projection data set. As is apparent from the above description, the data of two consecutive measurements can be combined, in any manner desired, with different magnification ratios within a specific sub-region of the object so as to provide high-resolution image data without impairing the image quality.

In other words, it is therefore possible to enable—from two or more recordings of the same object with different geometric magnifications—reconstruction of large measuring regions with such a high spatial resolution as cannot be achieved with a single measurement (multi-scan CT, multi-scan ROI CT).

Although the inventive embodiments have been discussed mainly in the context of material tests, it is readily possible to apply them to diagnosing methods in humans. When used for non-destructive testing of materials, the method may be used, in particular, for testing large components that have been produced by means of any production methods and from any materials for which, in their inner sub-regions, particularly high demands are placed on the spatial resolution that is effective in the testing. As an example, one might mention the testing of light metal casting pistons or turbine screws for power plants or generators. The testing precision is greatly improved by the above-described concept, both with regard to the sensitivity to cracks, to voids and pores, and with regard to the specificity in separating correct and erroneous positive defect findings.

In addition, applying the inventive concept enables simultaneously, i.e. with only one single CT construction, testing the further regions of the object with a low spatial resolution, it being possible to supplement the testing with the low spatial resolution by further testing by means of a method with a lower penetration depth.

Depending on the circumstances, the inventive method for CT reconstruction of an object may be implemented in hardware or in software. The implementation may be on a digital storage medium, in particular a disc or a CD having electronically readable control signals which can cooperate with a programmable computer system such that the inventive method for CT reconstruction of an object is performed. Generally, the inventive thus also consists in a computer program product having a program code, stored on a machine-readable carrier, for performing the inventive method, when the computer program product runs on a computer. In other words, the invention may thus be realized as a computer program having a program code for performing the method, when the computer program runs on a computer.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1-21. (canceled)

22. A method of producing a CT reconstruction of an object comprising a high-resolution object region of interest, the method comprising:
producing a first projection data set of a first region of the object that encloses the object region of interest and comprises at least one projection recording of a first resolution;
producing a second projection data set for the object region of interest that comprises at least one second projection recording of a second, higher resolution; pre-transforming the second projection data set to acquire a pre-transformed second projection data set comprising, within the object region of interest, only image frequencies above a predetermined cutoff frequency, which is equal to or smaller than an upper imaging frequency, defined by the first resolution, within the first projection data set; and
combining the first and the pre-transformed second projection data sets in accordance with a combination rule so as to acquire a CT reconstruction of the first region of the object comprising the first resolution, and of the object region of interest comprising the second, higher resolution.

23. The method as claimed in claim 22, wherein producing the first projection data set comprises imaging the entire object.

24. The method as claimed in claim 22, wherein
the first projection data set comprising a first magnification defined by a relative position between a detector and the object is recorded by means of the detector; and
the second projection data set comprising a second magnification defined by a second relative position between the detector and the object is produced by means of the detector, the second magnification being higher than the first magnification.

25. The method as claimed in claim 22, wherein
the first projection data set is produced by means of a detector with a first spatial resolution; and
the second projection data set is produced by means of a second detector with a second, higher spatial resolution.

26. The method as claimed in claim 22, wherein
the first projection data set is produced with X-radiation of a first X-ray energy; and
the second projection data set is produced with X-radiation of a second X-ray energy, the energy of which is predetermined such that the X-ray absorption of the material of the object leads, when using the second X-ray energy, to a projection recording whose contrast is larger or small than the contrast of the first projection recording.

27. The method as claimed in claim 22, wherein in combining, a third projection data set of the region enclosing the object region of interest is produced from the first projection data set and the pre-transformed second projection data set, said region enclosing the object region of interest comprising, within the object region of interest, image frequencies above and below the predetermined cutoff frequency.

28. The method as claimed in claim 27, wherein the third projection data set is acquired by a wavelet synthesis of the first projection data set and of the pre-transformed second projection data set.

29. The method as claimed in claim 27, wherein, in accordance with the combination rule, the CT reconstruction is produced from the third projection data set while using a CT reconstruction algorithm.

30. The method as claimed in claim 22, wherein in combining, in accordance with the combination rule, a first intermediate CT reconstruction is produced from the first projection data set, and a second intermediate CT reconstruction is produced from the pre-transformed second projection data set while using a CT reconstruction algorithm.

31. The method as claimed in claim 30, wherein the CT reconstruction is acquired by combining the first intermediate CT reconstruction and the second intermediate CT reconstruction.

32. The method as claimed in claim 31, wherein the combination of the first intermediate CT reconstruction and the second intermediate CT reconstruction is effected by means of a wavelet synthesis.

33. The method as claimed in claim 22, wherein the pre-transformed second projection data set produced by the pre-transformation corresponds to a wavelet representation of a CT reconstruction of the object region of interest.

34. The method as claimed in claim 22, additionally comprising:
producing one or more further projection data sets comprising a higher resolution than the first resolution for one or more further object regions of interest; and
combining the first and the one or several further projection data sets in accordance with the combination rule.

35. A device for producing a CT reconstruction of an object comprising a high-resolution object region of interest, the method comprising:
a detector configured to
produce a first projection data set of a first region of the object that encloses the object region of interest and comprises at least one projection recording of a first resolution;
produce a second projection data set of an object region of interest comprising at least a second projection recording of a second, higher resolution; and
a combiner configured to pre-transform the second projection data set to acquire a pre-transformed second projection data set comprising, within the object region of interest, only image frequencies above a predetermined cutoff frequency, which is equal to or smaller than an upper imaging frequency, defined by the first resolution, within the first projection data set, and to combine the first and the pre-transformed second projection data sets in accordance with a combination rule so as to acquire a CT reconstruction of the first region of the object comprising the first resolution, and of the object region of interest comprising the second, higher resolution.

36. The device as claimed in claim 35, wherein the detector is configured to image the entire object when producing the first projection data set.

37. The device as claimed in claim 35, wherein the detector is configured such that
the first projection data set comprising a first magnification defined by a relative position between a detector and the object is recorded by means of the detector; and
the second projection data set comprising a second magnification defined by a second relative position between the detector the object is produced by means of the detector, the second magnification being higher than the first magnification.

38. The device as claimed in claim 35, wherein the detector is configured such that
the first projection data set is produced by means of a detector with a first spatial resolution; and
the second projection data set is produced by means of a second detector with a second, higher spatial resolution.

39. The device as claimed in claim 35, wherein the detector is configured such that
the first projection data set is produced with X-radiation of a first X-ray energy; and
the second projection data set is produced with X-radiation of a second X-ray energy, the energy of which is predetermined such that the X-ray absorption of the material of the object leads, when using the second X-ray energy, to a projection recording whose contrast is larger or small than the contrast of the first projection recording.

40. The device as claimed in claim 35, wherein the combiner is configured such that the pre-transformed second projection recording produced by the pre-transformation corresponds to a wavelet representation of a CT reconstruction of the object region of interest.

41. The device as claimed in claim 35, wherein the detector is configured to produce one or more further projection data sets comprising a higher resolution than the second resolution for one or more further object regions of interest; and

42. A tangible computer readable medium including a computer program comprising program code for performing, when the computer program is executed by a computer, a method of producing a CT reconstruction of an object comprising a high-resolution object region of interest, the method comprising:
producing a first projection data set of a first region of the object that encloses the object region of interest and comprises at least one projection recording of a first resolution;
producing a second projection data set for the object region of interest that comprises at least one second projection recording of a second, higher resolution;
pre-transforming the second projection data set to acquire a pre-transformed second projection data set comprising, within the object region of interest, only image frequencies above a predetermined cutoff frequency, which is equal to or smaller than an upper imaging frequency, defined by the first resolution, within the first projection data set; and
combining the first and the pre-transformed second projection data sets in accordance with a combination rule so as to acquire a CT reconstruction of the first region of the object comprising the first resolution, and of the object region of interest comprising the second, higher resolution.
